(19) United States
(12) Patent Application Publication
(10) Pub. No.: US 2004/0023226 A1
(43) Pub. Date: Feb. 5, 2004

(54) Method for immobilizing nucleic acids

(76) Inventor: Derva Ozkan, Berlin (DE)

Correspondence Address: MCGLEW & TUTTLE, PC, SCARBOROUGH STATION, SCARBOROUGH, NY, 10510, US

(21) Appl. No.: 10/220,331

(22) PCT Filed: Feb. 27, 2001
(86) PCT No.: PCT/DE01/00812

(30) Foreign Application Priority Data
Feb. 28, 2000 (DE) 100 10 376.6
Oct. 20, 2000 (DE) 100 53 393.0

(51) Int. Cl. C12Q 1/68; C12M 1/34; B05D 3/00

(52) U.S. Cl. 435/6; 435/287.2; 427/2.11

(57) ABSTRACT

The invention relates to a method for immobilizing nucleic acids on one of the surfaces of a non-porous organic polymeric material which does not carry any primary and/or secondary amine groups. The inventive method comprises the following steps: (a) an aqueous solution containing a nucleic acid as well as a dissolved salt is prepared, whereby the cation of the salt is selected from the group comprised of sodium, magnesium and of mixtures of these cations; (b) the solution mentioned in step (a) is brought into contact with the surface of the polymeric material, and; (c) the surface of the polymeric material, which is in contact with the solution, is irradiated with UV light after step (b) or simultaneously thereto.

### FIELD OF THE INVENTION

The invention relates to a method for immobilizing nucleic acids on a surface of an organic polymeric material, comprising the following steps: a) an aqueous solution containing a nucleic acid is prepared, b) the solution mentioned in step a) is brought into contact with the surface of the polymeric material, c) the surface of the polymeric material, which is in contact with the solution, is irradiated with UV light after step b) or simultaneously thereto, to an immobilized system obtainable by means of such a method, and to the use of such an immobilized system.

### BACKGROUND OF THE INVENTION AND PRIOR ART

In many sectors of biochemistry, it is necessary to immobilize nucleic acids, in particular short oligonucleotides with 20 to 200 nucleotides, by binding to a substrate. A typical application range is given by the so-called nucleic acid chips, wherein various nucleic acids are bound in different areals, with corresponding association, of a surface of a substrate or carrier component. A binding of a nucleic acid to a surface of a solid body can in principle be made adsorptively, by physisorption or chemisorption. Even more desirable is however a covalent binding, since the latter is comparatively stable and reliably prevents a disturbing washing-off of bound nucleic acids in subsequent processing or analysis steps under usual conditions.

The document Nagata et al., FEBS Letters 183: 379-382, 1985, describes the adsorption of cloned DNA wit approx. 40 kB of polystyrene. PBS (8 mM Na₂PO₄, 1.5 mM KH₂PO₄, 137 mM NaCl, 2.7 mM KCl) with 0.1 M MgCl₂ was used, and only after aspiration irradiated with UV. Similar conditions were described in the documents FR 2663040, US 5510085 and Nikiforov et al., PCR Methods Applic. 3:285-291, 1994. The document Koehler et al., J. Clin. Microbiol. 29:638-641, 1991, describes the adsorption of phages DNA with approx. 7 kB at plastic materials. The document Nikiforov et al., Nucleic Acids Res. 22:4167-4175, 1994 describes the adsorption of oligonucleotides by a binding mediated by cationic detergents. In this prior art, the nucleic acids are typically longer DNA fragments with in most cases substantially more than a couple of kB.

Chemically activated substrate surfaces of polymeric materials, including polystyrene, and biologically active substances suitable for a covalent binding thereto are for instance described in the documents U.S. Pat. No. 4,736,019, US 4,657,873, US 4,654,299, US 4,419,444 and US 4,081,329. A chemical functionalization of polystyrene is also described in, the documents U.S. Pat. No. 3,956,219, US 3,886,486, US 3,974,110, US 3,995,094 and US 4,226,958.

The document Church et al., Proc. Natl. Acad. Sci. USA 81:1991-1995, 1984, describes a photo-chemical method for cross-linking genomic DNA fragments on nylon membranes. From the document Saito et al., Tetrahedron Lett., 22: 3265-3268, 1981, it is known in the art that primary amine groups are highly reactive with light-activated thymidine, and this process is assumed to be a basis of the mechanism of the covalent binding of nucleotides to membranes. The document WO 8911548 describes a method and reagents for immobilizing oligonucleotides by a binding of a polynucleotide, preferably of a spacer of poly-dT, to a nucleic acid sample and fixing of the spacer to a substrate with primary and secondary amine groups, for instance nylon, by means of UV irradiation. The spacer is longer than the nucleic acid sample.

For the prior art measures it is regularly required to perform an activation of the surface of an organic polymeric material for a reaction with an oligonucleotide and/or to use a polymeric material carrying primary and/or secondary amine groups. The first condition mentioned is disturbingly expensive. In either case, further the remaining reactivity due to not bound-off reactive surface sites will be disturbing. Furthermore, in the prior art measures, there are in most cases long DNA fragments being immobilizable, whereas short nucleic acids obviously are not adsorbed under comparable conditions. Finally, the prior art measures offer an adsorptive binding only that is not sufficiently stable for various applications. Technical object of the invention.

The invention is based on the technical object to specify a method for producing an immobilized system with nucleic acids, in particular oligonucleotides, firmly bound to a polymeric material, said immobilized system not requiring an activation of the surface of the material by means of activation reagents or the like, nor a material per se reactive by primary and/or secondary amine groups.

### BASICS OF THE INVENTION

For achieving this technical object, the invention teaches a method for immobilizing nucleic acids with less than 300 base pairs on one of the surfaces of a non-porous polymeric material which does not carry any primary and/or secondary amine groups, comprising the following steps: a) an aqueous solution containing a nucleic acid as well as a dissolved salt is prepared, b) the solution mentioned in step a) is brought into contact with the surface of the polymeric material, c) the surface of the polymeric material, which is in contact with the solution, is irradiated with UV light. After step c), the nucleic acids are immobilized, and the solution can be drawn off. The step c) can be performed simultaneously or immediately after step b). If the step c) is performed immediately after step b), this means for instance a manipulation-caused time span typically being less than 30 min, in most cases less than 5 min, better less than 1 min. The nucleic acid may have less than 200, in particular less than 100 base pairs.

Surprisingly, the immobilization is successful on the surfaces of polymeric materials, and that on surfaces not having been chemically or physically activated before, alone by mediation of the salt present in the solution containing the nucleic acid in conjunction with UV light irradiation, without an adsorption step and/or aspiration being made before. Obviously a direct, non-adsorptive immobilization takes place. The nucleic acid cannot be removed from the surface anymore after the immobilization under the conditions being typical for the processing and/or analysis. The binding of the nucleic acid to the surface is obviously covalent.

The invention further teaches an immobilized system with a polymeric material and a nucleic acid bound to a surface of the polymeric material, obtainable by means of the method according to the invention, the surface being planar or being the outer surface of a polymeric fiber, and the use of such an immobilized system in a method for hybridizing nucleic acids, wherein the nucleic acid immobilized on the surface of the polymeric material comprises a hybridization region, comprising the following steps: a) the surface of the immobilized system carrying the nucleic acid is treated with a solution promoting the wetting of the surface, b) a part of the surface of the immobilized system carrying the nucleic acid is then brought into contact with a hybridization solution, said hybridization solution automatically and completely wetting the surface of the immobilized system carrying the nucleic acid. In the wetted region then the hybridization and possibly the detection thereof takes place in a conventional way. In the latter aspect of the invention, a detergent and a salt are bound to the surface of the immobilized system (for instance by block step). The hybridization solution contains targets and has the peculiarity that it contains very few ions (for instance less than 100 mM salt total concentration) and further is alkalic (pH 8-14). Thereby double-stranded DNA/RNA targets exist as a single strand which will only become binding by the action of the detergents and/or salts bound to the surface of the immobilized system. With the used ion strengths of the hybridization solution, a hybridization will namely per se not occur. In this context, it is equally important that the hybridization solution is applied in a small volume only and will distribute as a capillary film along the surface.

The above mentioned use is, referred to the application of a solution promoting the wetting, of an independent importance, i.e. independent from the use of the immobilized system according to the invention. The use of a solution promoting the wetting of a surface in conjunction with immobilized systems can namely in principle take place for any immobilized systems containing biologically active substances or material, thus also for instance such with proteins, peptides and/or sugars. In any case it is achieved that a solution added to the immobilized system containing a binding partner or prospective binding partner for the immobilized biologically active material will automatically distribute practically over the surface of the immobilized system and form a capillary film. Thereby, a smaller amount of the solution to be added is required. Further, when using a same amount of a compound in the added solution, a stronger signal is obtained in case of a binding, since the ratio border area solution/immobilized system to volume of the added solution is increased, due to the generation of a capillary film. The analytical performance will thus become more sensitive.

Immobilized systems according to the invention can be used for the actual analysis (analysate characterizing) as well as for the preparation of a sample, for instance for separating specific analysate components by solid-phase binding from a complex mixture, e.g. of genomic DNA from blood samples or RNA etc or for instance for enrichment at the solid phase, e.g. by enzymatic methods. For the latter, for instance a binding site for an enzyme, such as T7 DNA polymerase, can be built in into the immobilized oligonucleotide.

### PREFERRED EMBODIMENTS OF THE INVENTION

After the step c), a washing step may be performed. Thereby, not (covalently) bound nucleic acid molecules are so to speak washed off or desorbed. After step c) or after the washing step, a rinsing step with an aqueous solution containing a detergent may be performed. By means of the detergent, any reactive surface sites having been kept unoccupied are saturated or blocked, but also the adhesion forces between a subsequently added solution and the immobilized system compared to the cohesion forces in the subsequently added solution are increased, and so the wetting capability of the immobilized system is increased, up to the complete self-wetting or automatic generation of a capillary film.

The polymeric material may be selected from the group comprised of “polyethylene (PE), polycarbonate (PC), polyvinyl chloride (PVC), polystyrene (PS), polyethylene terephthalate (PETP), polyethersulfone (PES), polyetherether ketone (PEEK), polyphenylene oxide (PPO), polyphenylene sulfide (PPS), polybutylene terephthalate (PBT), polyoxymethylene (POM), polysulfone (PSU), polyetherimide (PEI), polyamide (PA) and mixtures and copolymers of the monomers of such polymers”, in particular selected from the group comprised of “polycarbonate (PC), polyvinylchloride (PVC), polystyrene (PS) and mixtures and copolymers of the monomers of such polymers”.

The concentration of the nucleic acid in the solution may be in the range from 0.0001 to 1,000 nM, preferably from 1 to 100 nM. It is understood that different nucleic acids may also be used, in concentrations as respectively specified.

The total concentration of the dissolved salt in the solution is suitably in the range from 0.1 to 6 M, preferably in the range from 0.1 to 3 M. The salt may be selected from the group comprised of “MgCl₂, CaCl₂, NaCl, KCl, LiCl, NH₄HCO₃, NaHCO₃, Na₂CO₃, NH₄Cl, NaHPO₄, NaPi, NH₄AC, NaAc, KAc, Tris HCl, HCl, Tris base, KHSO₄, K₂S₂O₅, tetraethylammonium chloride, MOPS, HEPES, succinic acid, diethanolamine, ethanolamine, NaHCO₃/NaCl/EDTA, PBS, TAE, bisulfite, Na borate and mixtures of these substances”. A salt is a substance dissociating in an aqueous solution into at least one anion and at least one cation. The UV light should have a wavelength in the range from 1 nm to 480 nm, in particular 100 nm to 300 nm. The time duration of the irradiation is typically 1 s to 100 min, preferably 10 s to 10 min, for instance 0.5 to 2 min. A radiation power such as provided by a 60 watts UV lamp over 2 min at a distance of 15 cm is fully sufficient.

The detergent may be selected from the group comprised of “Tween 20, Triton X100, Brij-35, sarkosyl and mixtures of such substances”.

In the embodiment of an immobilized system according to the invention, wherein the surface is the outer surface of a polymeric fiber, a plurality of such polymeric fibers are arranged and fixed in parallel or at a distance from each other that increases in the longitudinal direction, the outer surfaces of the adjacent polymeric fibers do not touch each other or only with point or line contact, and the interspaces between the polymeric fibers are so tight that in the interspaces occurs a capillary ascension of subsequently added solutions of at least 0.1 mm.

The solution promoting the wetting may contain a detergent selected from the group as described in the previous section. The hybridization solution has preferably a pH of 7 to 9. The adjustment of the pH may be performed by using NaOH.

The invention may be employed, in addition to the usual planar biochips, in particular in conjunction with special components of the following structure.

The special component comprises a plurality of fiber elements (fibers) and sample molecules being immobilized on the fiber elements of selected sample molecule species or selected sample molecule species groups, to each fiber element being associated a specific sample molecule species or sample molecule species group, and is characterized by that the sample molecules are immobilized on cylindrical outer surfaces of the fiber elements, and that the fiber elements are fixed by means of a supporting element in a radial direction with regard to the fiber elements in an interspaced manner or are bundled together with linear contact. The fiber elements may be arranged in parallel to each other as a fiber element bundle. The fiber elements may be optical fiber elements. The front faces of at least one end of the fiber elements, preferably of both ends, are optically contactable. Alternatively or additionally, an electrical contacting is possible, for instance for measuring impedances or impedance changes. Evaluations by detection of surface plasmon resonances or scattering processes are also possible. The fiber elements may have a diameter in the range from 0.01 μm to 1,000 μm and/or a length in the range from 0.1 μm to 100 mm. The fiber elements may be packed in a density of 1 to 10 fibers/cm, referred to a radial cross-sectional plane of the fiber elements. The ratio diameter to length may be in the range from 100 to 10. The supporting element may be arranged at one end of the fiber element and structured so to enclose the ends of the fiber elements, the front faces of the enclosed fiber elements being directly or indirectly optically and/or electrically contactable. One supporting element each may be arranged at both ends of the fiber elements. A supporting element may however also be arranged between the two ends of the fiber elements, for instance centrally. The supporting element may be adapted as a perforated plate or as a wound-up supporting ribbon. The supporting element may finally be formed from sections of the fiber elements, by welding, melting, gluing or the like. The sample molecule species or the sample molecule species groups may be selected from the group comprised of “DNA, RNA, PNA and mixtures of these nucleic acids”. Every fiber element may carry sample molecules of a respectively selected, different sample molecule species. On a fiber element, different fields with different nucleic acid species may be provided. Every fiber element may however also carry sample molecules of a respectively selected, different sample molecule species group, the group elements of each sample molecule species group commonly binding under creation of co-operative effects to a defined target molecule. Every sample molecule species group may contain two group elements. Such a special component may be produced in a method comprising the following steps: a) a plurality of endless fibers are produced, b) each endless fiber is guided through a fluid containing a selected sample molecule species or a selected sample molecule species group, c) the sample molecules of the sample molecule species or sample molecule species group are immobilized (according to the invention) on the endless fibers, d) as an option the endless fibers are supplied to at least one washing step, e) to each endless fiber is directly or indirectly associated the sample molecule species or sample molecule species group immobilized on the fiber in step c), f) from different endless fibers, one fiber element each is cut off, and the fiber elements of different endless fibers are connected to a supporting element or are bundled and fixed together in a linear contact. The association may take place by interposition of a coding of the endless fibers for instance by mixing-in quantum spots or dyes in the raw mass of the endless fibers prior to the extrusion. Then results the association of the nucleic acid species/group to the position of a fiber element in a completed component only after position-resolved determination of the coding. Thereby an exact positioning of the fiber elements during the production of the component is obsolete. Such components can for instance be used in a method for the detection of target molecules, wherein optically contactable front faces of the fiber elements are optically connected for instance to a CCD array or by a micro-mirror system to a photomultiplier being sensitive to optical radiation of the wavelength to be detected, and wherein sensor elements of the CCD array or micro-mirror or micro-mirror positions are each associated to the fiber elements, comprising the following steps: a) to the biochip is supplied a solution with prospective target molecules, under conditions at which target molecules bind to sample molecules, b) simultaneously with step a) or subsequently thereto the biochip is irradiated with a primary radiation exciting the wavelength to be detected, c) simultaneously with step b) or subsequently thereto a reading-out of the signals of the sensor elements of the CCD array or of the photomultiplier and processing and storage of the signals is performed.

Definitions.

A component is a device carrying sample molecules of a sample molecule species or sample molecule species group in discrete and defined surface regions, the sample molecule fields. Normally every sample molecule field of a component will carry a different sample molecule species or sample molecule species group. The sample molecule fields are addressable in the sense that a direct or indirect association is/has been made between every sample molecule field or its geometric position in the component and the sample molecule species group carried by the sample molecule field.

Sample molecules are molecules that can enter a specific interaction with target molecules. Examples for such interactions are: protein-aptamer, nucleic acid-nucleic acid (Crick/Watson or not Crick/Watson), nucleic acid-ribozyme, etc.

Target molecules are molecules for which a sample to be analyzed and supplied to the biochip is examined.

A sample molecule species includes sample molecules of exclusively one structure, for instance of one sequence in the case of nuclear acids or proteins or peptides.

A sample molecule species group includes as group elements at least two sample molecule species. Sample molecule species may be identical or different sample molecule types.

Co-operative effects between molecules of several sample molecule species or elements of a sample molecule group and a target molecule species are characterized by that the energy gain by simultaneous interaction between respectively the molecules of the different sample molecule species on one hand and that between the different sample molecule species and the target molecule as a whole on the other hand is greater than the sum of the energy gains of the interactions of a molecule of respectively one sample molecule species with one molecule of the target molecule species. The additional energy gain thus originates from the co-operation between the sample molecule species in the case of a binding of the sample molecule with the target molecule. In the case of the nucleic acids as sample molecules and target molecules are for instance to be named stacking effects. The stacking effect is an energy gain by interactions, namely delocalization of the ¶ electrons of the hydrophobic ring structures of adjacent bases in double-stranded nucleic acids. The stacking effect occurs between the ends of the sample nucleic acids, if a binding to a target molecule takes place in such a manner that the ends of the sample nucleic acids are bound adjacently to each other. In the case of the proteins, co-operative effects may result from special secondary structures of proteins interacting with each other. Generally, a higher specificity of a binding between sample molecules and a target molecule is achieved with co-operative effects.

A hybridization region is a sequence region of a sample nucleic acid being able to hybridize with a target nucleic acid. A spacer region is a group bound to one end of the sample nucleic acid and bound by a second binding site to the sample molecule field. A spacer region suitably is configured so that a binding or hybridization with a target molecule cannot take place. A spacer region may for instance be formed from a non-hybridizing oligonucleotide. By a spacer region it is achieved that on one hand the hybridization region is arranged at a sufficient distance to the surface of the sample molecule field and on the other hand the hybridization region is practically flexibilized and thus can bind to a target molecule without steric or conformation-caused restrictions.

As fiber elements are designated pieces cut off from an endless fiber. Normally, the cut will be made in a plane orthogonally to the longitudinal extension of the endless fiber, however, of course, a cutting plane at an angle less than 90° is also possible.

An endless fiber is a rod-type or thread-type structure having a large longitudinal extension compared to the length of the fiber elements, typically produced by means of drawing technologies, blowing technologies and/or extrusion technologies and wound up and stored on drums or the like.

An endless fiber and/or a fiber element may comprise in a cross-sectional plane orthogonally to the longitudinal extension the most various cross-sectional shapes. Just preferred is a substantially circular cross-section. In so far the term cylindrical outer surface comprises, for the purpose of the invention, also outer surfaces in the case of non-circular cross-sections. In so far the term of the radial direction designates, for the purpose of the invention, all directions in a cross-sectional plane. In so far finally the diameter, for the purpose of the invention, is d=2·(F/2¶)^{0.5}, F being the cross-sectional area (any shape).

The front face of a fiber element is formed by a cut through an endless fiber.

Spacing of the fiber elements means that the outer surfaces of adjacent fiber elements do not touch each other or are supported against each other by noses. The contact areas are at any case less than 1% of the total area of the fiber elements.

A fiber element bundle usually comprises mutually coplanar front faces of the bundled fiber elements. It is however also possible to adapt within a fiber element bundle groups of fiber elements with respectively coplanar front faces, the front faces of fiber elements of different groups not being mutually coplanar.

Optical fiber elements are optically transparent for electromagnetic radiation, at least in a partial section of the ranges IR, visible light and/or UV. Optically transparent means that the attenuation of the electromagnetic radiation is sufficiently low in order to permit a detection of electromagnetic radiation produced at one end of a fiber element at the opposite end of the fiber element by means of usual detection technologies.

The term optical contactability designates a treatment of a partial face of a fiber element permitting the emission of electromagnetic radiation out of the fiber element through the partial face. A strong scattering should be prevented, if possible. It could be of help to machine the partial faces in a suitable way, for instance smooth or polish.

A non-porous material has a degree of porosity (open porosity), measurable by means of the xylol method, of less than 1%, preferably less than 0.1%, even better less than 0.01% (volume of pores/total volume workpiece).

Detergents are substances reducing the stresses at borderlines, in particular tensides, ionic, non-ionic or ampholytic.

A solution promoting the wetting of a surface typically contains one or more detergents. Whether a solution meets the criterion of promoting the wetting, can be detected by that two defined capillaries are produced from the polymeric material, one of the capillaries being treated with the solution, the second one however not. Then, for both capillaries, the capillary ascension is determined for instance with a hybridization solution under the same conditions. If the capillary ascension in the treated capillary is greater than in the untreated one, the used solution promotes the wetting.

In the following, the invention is explained in more detail, based on not limiting examples of execution.

### EXAMPLE 1

### Immobilization of a Nucleic Acid

An aqueous solution containing an oligonucleotide with the sequence (T)20-ATT CTA GCT AGT TCA ACT TC (10 nM), MgCl₂ (0.2 M), NaCl (1.0 M) and Tris (0.1 M) was prepared, a pH of 8 being adjusted. 1 μl of this solution was brought on a polycarbonate platelet, and the polycarbonate platelet with the applied solution was irradiated for 1 min with UV light of the wavelength 254 nm. The platelet was then rinsed with a detergent-containing solution.

### EXAMPLE 2

### Immobilization of a Nucleic Acid (Reference Example)

Same procedure as in example 1, with the difference that an irradiation with UV light was not made.

### EXAMPLE 3

### Hybridization of the Immobilized Systems of Examples 1 and 2

The platelets were each brought into a hybridization solution containing a biotin-marked oligonucleotide with the sequence GCT GAA ATG GCA ATG GAA GTT GAA CTA GCT, and an incubation was made for 10 min at 20° C. Then a rinsing step with a solution corresponding to the hybridization solution was performed, however without the oligonucleotide. Then an examination for hybridization was made by means of a streptavidin peroxidase conjugate and a colorimetric substrate. The platelet of example 1 shows a coloration proving the coupling of the oligonucleotide with the measures of the example 1. The platelet of example 2 did however not show a coloration; a binding of the oligonucleotide to the surface has therefor not taken place without irradiation.

### EXAMPLE 4

### Examination of the Binding Stability of the Oligonucleotide for the Platelet of Example 1

Tests for separating the oligonucleotide from the surface of the platelet with solutions containing SDS, 1 M NaOH and/or chaotropic reagents at 100° C. and for 2 h in an aqueous solution showed that the oligonucleotide remains firmly bound. Such conditions would be suitable to abolish non-covalent interactions between the oligonucleotide and the platelet.

### EXAMPLE 5

### Tests with Other Polymeric Materials

The tests of examples 1 to 4 were repeated with the difference that the platelet was made from polystyrene or polyvinylchloride rather than from polycarbonate. These two polymers do at any case not contain primary or secondary amine groups. In all tests, results were obtained corresponding to the examples 1 to 4.

### EXAMPLE 6

### Use of a Solution Promoting the Self-Wetting Feature

An immobilized system according to example 1 was prepared, with the difference that the polymeric material was polystyrene and was formed to a micro test plate (MTP). The MTP was then rinsed with a solution promoting the self-wetting feature containing NaCl (0.75 M), sodium citrate (0.075 M), Tween® 20 (0.05 wt. % referred to the total weight solution), and as an option sodium azide (0.05 wt. % referred to the total weight solution).

Then for hybridization in a capillary film, 5 μl of a 10 mM NaOH containing 1 μM of a biotin-marked oligonucleotide with the sequence GCT GAA ATG GCA ATG GAA GTT GAA CTA GCT were brought into the recess of the MTP.

Due to the combination surface-bound detergent and slightly alkalic hybridization solution, not only a wetting of the complete recess of the MTP by the capillary film took place, but simultaneously a stabilization and hybridization of the target at the immobilized nucleic acids. After 15 min incubation at 20° C., a hybridization could be detected by means of a streptavidin peroxidase conjugate and a colorimetric substrate.

Reference recesses provided with a higher volume (10 to 200 μl) showed a decreasing signal strength.

Further tests showed that the binding of detergents to the surface is important, on one hand for generating a capillary film and on the other hand in order to cause a stabilization/hybridization of previously denaturated nucleic acids directly at the surface. A nucleic acid being present in a denaturated condition in a neutral or alkalic solution (corresponding to 1 to 200 mM NaOH) is thus made hybridizable exclusively in case of a contact with the surface.

### EXAMPLE 7

### Immobilization with Different Salts at Different Concentrations

A binding was detected for the following salts at different pH values: MgCl₂, CaCl₂, NaCl, KCl, LiCl, NH₄HCO₃, NaHCO₃, Na₂CO₃, NH₄Cl, NaHPO₄, NaPi, NH₄Ac, NaAc, KAc, Tris HCl, HCl, Tris base, KHSO₄, K₂S205, tetraethylammonium chloride, MOPS, HEPES, succinic acid, diethanolamine, ethanolamine, NaHCO₃/NaCl/EDTA, PBS, TAE, bisulfite, Na borate. Therein the concentration has been adjusted to 100 mM.

Some of these salts were investigated for a concentration dependency. NaCl, NH₄Cl and NaPi caused 100% binding down to 10 mM, at 5 mM 80% binding and at 1 mM and less 0% binding. NaHCO₃ had the same behavior, with only 60% binding at 5 mM. MgCl₂ showed 100% binding down to 10 mM, followed by 80% binding down to 0.1 mM. KHSO₄ showed 100% binding down to 5 mM, 80% binding at 1 mM and 0.5 mM and 60% binding at 0.1 mM. NH₄Ac showed 100% binding down to 10 mM, 80% binding down to 1 mM and 40% binding down to 0.1 mM. It can be assumed that the other salts also show down to 5 mM at least 80% binding, at any case 100% binding at 10 mM.

1. A method for immobilizing nucleic acids with less than 300 base pairs on one of the surfaces of an organic polymeric material which does not carry any primary and/or secondary amine groups, comprising the following steps:
a) an aqueous solution containing a nucleic acid as well as a dissolved salt is prepared,
b) the solution mentioned in step a) is brought into contact with the surface of the polymeric material,
c) the surface of the polymeric material, which is in contact with the solution, is irradiated with UV light.

2. A method according to claim 1, wherein after the step c), a washing step is performed.

3. A method according to claim 1 or 2, wherein after the step c) or after the washing step, a rinsing step with an aqueous solution containing a detergent is performed.

4. A method according to one of claims 1 to 3, wherein the polymeric material is selected from the group comprised of “polyethylene (PE), polycarbonate (PC), polyvinyl chloride (PVC), polystyrene (PS), polyethylene terephthalate (PETP), polyethersulfone (PES), polyetherether ketone (PEEK), polyphenylene oxide (PPO), polyphenylene sulfide (PPS), polybutylene terephthalate (PBT), polyoxymethylene (POM), polysulfone (PSU), polyetherimide (PEI), polyamide (PA) and mixtures and copolymers of the monomers of such polymers”, in particular selected from the group comprised of “polycarbonate (PC), polyvinylchloride (PVC), polystyrene and mixtures and copolymers of the monomers of such polymers”.

5. A method according to one of claims 1 to 4, wherein the concentration of the nucleic acid in the solution is in the range from 0.0001 to 1,000 nM, preferably from 1 to 100 nM.

6. A method according to one of claims 1 to 5, wherein the total concentration of the dissolved salt in the solution is in the range from 0.1 mM to 6 M, preferably in the range from 0.1 to 3 M, in particular from 0.1 to 0.2 M.

7. A method according to one of claims 1 to 6, wherein the salt is selected from the group comprised of “MgCl₂, CaCl₂, NaCl, KCl, LiCl, NH₄HCO₃, NaHCO₃, Na₂CO₃, NH₄Cl, NaHPO₄, NaPi, NH₄AC, NaAc, KAc, Tris HCl, HCl, Tris base, KHSO₄, K₂S₂O₅, tetraethylammonium chloride, MOPS, HEPES, succinic acid, diethanolamine, ethanolamine, NaHCO₃/NaCl/EDTA, PBS, TAE, bisulfite, Na borate and mixtures of these substances”.

8. A method according to one of claims 1 to 7, wherein the UV light has a wavelength in the range from 1 nm to 480 nm, in particular 100 nm to 300 nm.

9. A method according to one of claims 3 to 8, wherein the detergent is selected from the group comprised of “Tween 20, Triton X100, Brij-35, sarkosyl and mixtures of such substances”.

10. An immobilized system comprising a polymeric material and a nucleic acid bound to the surface of the polymeric material, obtainable by a method according to one of claims 1 to 9, wherein the surface is planar or is the outer surface of a polymeric fiber.

11. The use of an immobilized system according to claim 10 in a method for hybridizing nucleic acids, wherein the nucleic acid immobilized on the surface of the polymeric material comprises a hybridization region, comprising the following steps:
a) the surface of the immobilized system carrying the nucleic acid is treated with a solution promoting the wetting of the surface,
b) a part of the surface of the immobilized system carrying the nucleic acid is then brought into contact with a hybridization solution, said hybridization solution automatically and completely wetting the surface of the immobilized system carrying the nucleic acid.

12. A method according to claim 11, wherein the detergent promoting the wetting is selected from the group comprised of “Tween 20, Triton X100, Brij-35, sarkosyl and mixtures of such substances”.

13. A method according to claim 11 or 12, wherein the hybridization solution has a pH value from 6.5 to 11, preferably 7 to 9.
